(19) United States
(12) Patent Application Publication
(10) Pub. No.: US 2021/0038681 A1
(43) Pub. Date: Feb. 11, 2021

(54) PHARMACEUTICAL COMPOSITION FOR TREATING VIRAL HEPATITIS C

(71) Applicant: ASCLETIS BIOSCIENCE CO., LTD., Hangzhou (CN)

(72) Inventors: Jinzi Jason Wu, Hangzhou (CN); Kristjan Gudmundsson, Hangzhou (CN); Bailing Yang, Hangzhou (CN); Bo Shan, Hangzhou (CN)

(21) Appl. No.: 16/965,667

(22) PCT Filed: Feb. 9, 2018
(86) PCT No.: PCT/CN2018/075916

§ 371 Date: Jul. 29, 2020

(30) Foreign Application Priority Data
Feb. 2, 2018 (CN) 201810106009.9

(51) Int. Cl. A61K 38/12; A61P 31/14

(52) U.S. Cl. CPC A61K 38/12; A61P 31/14

(57) ABSTRACT

The present invention provides a pharmaceutical composition for treating viral hepatitis C, comprising ravidasvir (ASC16) or a pharmaceutically acceptable salt thereof and danoprevir (ASC08) or a pharmaceutically acceptable salt thereof. The combined use of ASC16 and ASC08 has an enhanced effect, and the experimental results show that ASC16 and ASC08 can form an effective combined anti-HCV treatment regimen. Moreover, the administration regimen of ASC16 in combination of ASC08 further expands the action spectrum of therapeutic drugs. More importantly, the combined use of ASC16 and ASC08 solves the problem of drug resistance.

### TECHNICAL FIELD OF THE INVENTION

The present invention relates to the technical field of medicine, in particular to a pharmaceutical composition for treating viral hepatitis C, and use of the pharmaceutical composition in the preparation of a medicament and a kit for treating viral hepatitis C.

### BACKGROUND OF THE INVENTION

Viral hepatitis C, abbreviated as hepatitis C, is a type of viral hepatitis caused by hepatitis C virus (HCV) infection, which is mainly transmitted through blood transfusion, acupuncture, drug abuse and other routes. According to the statistics from the World Health Organization, the global infection rate of hepatitis C virus is about 3%, and it is estimated that about 180 million people have been infected with hepatitis C virus, and about 35,000 new cases occur each year. Hepatitis C can cause chronic inflammatory necrosis and fibrosis of the liver, and some patients may develop liver cirrhosis or even hepatocellular carcinoma (HCC). Hepatitis C is extremely harmful to the health and lives of patients, and has become a serious social and public health problem.

Ravidasvir (code name ASC16) is a therapeutic drug for hepatitis C developed by the Ascletis Bioscience Co., Ltd. It is a potent pan-genotypic HCV NS5A inhibitor, which can selectively inhibit multifunctional HCV NS5A protein. The clinical trials have shown that ravidasvir is an effective therapeutic drug for hepatitis C.

Since viral RNA replicase is prone to errors during the virus replication, drug-resistant variants may have already existed and/or appear quickly in the presence of an inhibitor. The appearance of drug-resistant viruses is the main challenge in developing effective antiviral treatment regimens for HCV infection. The appearance of drug-resistant variants for ravidasvir has caused problems in the treatment of ravidasvir against HCV infection.

### SUMMARY OF THE INVENTION

In order to solve the problem of existence of HCV drug-resistant variants in the prior art, the present invention conducted an in-depth and extensive research on the antiviral activity and antiviral spectrum of pharmaceutical compositions comprising ravidasvir and other active ingredients against hepatitis C virus, thereby providing a pharmaceutical composition for effectively treating viral hepatitis C, especially effectively inhibiting ravidasvir-resistant variants.

Danoprevir (DNV, code name ASC08) is a macrocyclic peptidomimetic compound, which belongs to the second-generation of protease inhibitors and competitively inhibits the activity of HCV NS3/4A protease. Compared with the first-generation of protease inhibitors, danoprevir has the characteristics of inhibition of multi-genotypic viruses, stronger antiviral activity, higher barrier to drug resistance, and better safety.

The pharmaceutical composition provided by the present invention comprises ravidasvir or a pharmaceutically acceptable salt thereof and danoprevir or a pharmaceutically acceptable salt thereof. Preferably, said pharmaceutically acceptable salt of ravidasvir is ravidasvir hydrochloride, and said pharmaceutically acceptable salt of danoprevir is danoprevir sodium.

In the pharmaceutical composition according to the present invention, the mass ratio of ravidasvir or a pharmaceutically acceptable salt thereof to danoprevir or a pharmaceutically acceptable salt thereof is 1:1.

Preferably, the pharmaceutical composition of the present invention further comprises ritonavir or a pharmaceutically acceptable salt thereof; more preferably, the mass ratio among ravidasvir or a pharmaceutically acceptable salt thereof, danoprevir or a pharmaceutically acceptable salt thereof, and ritonavir or a pharmaceutically acceptable salt thereof is 1:1:1.

Preferably, the pharmaceutical composition of the present invention further comprises a pharmaceutically acceptable excipient, for example, said pharmaceutically acceptable excipient is selected from one or more of poloxamer 188, mannitol 160C, mannitol 200SD, microcrystalline cellulose (PH101), isomalt, Span 20, or polyvinylpyrrolidone (VA64).

Preferably, the pharmaceutical composition of the present invention is a film-coated tablet.

According to one embodiment of the present invention, in the pharmaceutical composition of the present invention, ravidasvir or a pharmaceutically acceptable salt thereof and danoprevir or a pharmaceutically acceptable salt thereof are placed separately.

More preferably, the pharmaceutical composition of the present invention further comprises ritonavir or a pharmaceutically acceptable salt thereof, which is placed separately.

According to one embodiment of the present invention, the administration dosages of danoprevir or a pharmaceutically acceptable salt thereof and ravidasvir or a pharmaceutically acceptable salt thereof are: ravidasvir or a pharmaceutically acceptable salt thereof, once a day, 200 mg; danoprevir or a pharmaceutically acceptable salt thereof, twice a day, 100 mg each time, respectively.

According to one embodiment of the present invention, the administration dosages of danoprevir or a pharmaceutically acceptable salt thereof, ravidasvir or a pharmaceutically acceptable salt thereof, and ritonavir or a pharmaceutically acceptable salt thereof are: ravidasvir or a pharmaceutically acceptable salt thereof, once a day, 200 mg; danoprevir or a pharmaceutically acceptable salt thereof, twice a day, 100 mg each time; ritonavir or a pharmaceutically acceptable salt thereof, twice a day, 100 mg each time, respectively.

Therefore, the present invention also provides a kit for treating viral hepatitis C, comprising one unit dose of ravidasvir or a pharmaceutically acceptable salt thereof, one unit dose of danoprevir or a pharmaceutically acceptable salt thereof, and another unit dose of danoprevir or a pharmaceutically acceptable salt thereof, which are placed separately. Preferably, said one unit dose of ravidasvir or a pharmaceutically acceptable salt thereof is 200 mg of ravidasvir or a pharmaceutically acceptable salt thereof, and said one unit dose of danoprevir or a pharmaceutically acceptable salt thereof and another unit dose of danoprevir or a pharmaceutically acceptable salt thereof are 100 mg of danoprevir or a pharmaceutically acceptable salt thereof, respectively; more preferably, said kit further comprises one unit dose of ritonavir or a pharmaceutically acceptable salt thereof and another unit dose of ritonavir or a pharmaceutically acceptable salt thereof, which are placed separately. Preferably, said one unit dose of ritonavir or a pharmaceutically acceptable salt thereof and another unit dose of ritonavir or a pharmaceutically acceptable salt thereof are 100 mg of ritonavir or a pharmaceutically acceptable salt thereof, respectively.

The present invention also provides use of the above-mentioned pharmaceutical compositions in the preparation of a therapeutic drug for hepatitis C.

The present invention also provides a method for treating hepatitis C, comprising administering a therapeutically effective amount of the above-mentioned pharmaceutical compositions to a subject in need thereof, or applying the above-mentioned kit to a subject in need thereof.

The study results of the present invention have confirmed that the combined use of ASC16 and ASC08 has an enhanced effect, indicating that ASC16 and ASC08 can form an effective combination anti-HCV treatment regimen. Moreover, the administration regimen of ASC16 in combination with ASC08 further expands the action spectrum of therapeutic drugs. More importantly, the combined use of ASC16 and ASC08 has solved the problem of resistance to ravidasvir. The phase II clinical trial showed that the overall cure rate (SVR12 rate) of administration regimen of ASC16 in combination with ASC08 was 100% (38/38 subjects), and the efficacy was not affected by the baseline resistance mutations of NS5A. All subjects showed a generally good tolerance after receiving the treatment, and the adverse events during the treatment were mostly mild or moderate, without severe adverse events, and no subjects withdrew from the treatment due to the adverse events. Therefore, the combination of ravidasvir and danoprevir of the present invention is an effective treatment regimen for hepatitis C, which is of great clinical and social significance.

### BRIEF DESCRIPTION OF THE DRAWINGS

Hereinafter, the embodiments of the present invention will be illustrated in detail with reference to the drawings, in which:

FIG. 1 shows the hepatitis C virus (HCV) replicon cell line.

### DETAILED DESCRIPTION OF THE INVENTION

In order to better illustrate the present invention and facilitate the understanding of the technical solutions of the present invention, the present invention will be further set forth with reference to the accompanying drawings and examples. It should be understood that the specific examples of the present invention are for illustrative purpose only, and not intended to limit the present invention.

### EXAMPLE 1

Non-Clinical Pharmacodynamic Studies

1.1 Antiviral Efficacy and Action Spectrum of ASC16 against HCV

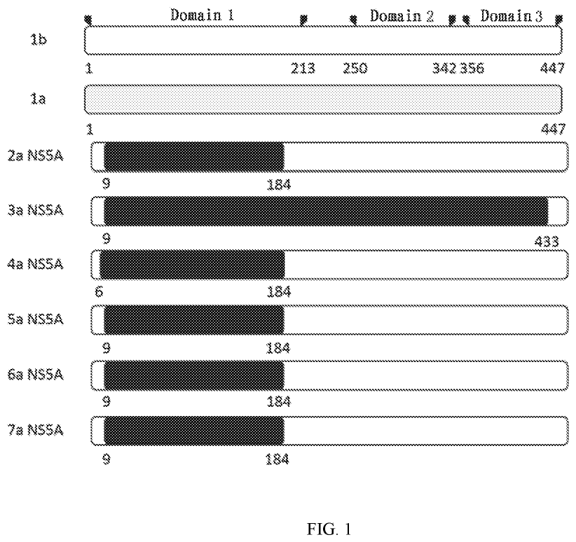

Wild-type HCV viruses are divided into 7 types of major genotypes. In order to evaluate the antiviral activities of ASC16 against all other major HCV genotypes except 1a and 1b, a stable HCV 1b chimera replicon cell line encoding the key fragments of NS5A genes for other HCV genotypes was created by replacing the coding region of HCV 1b NS5A with amino acids 9-184 of genotype 2a, 5a or 6a, amino acids 9-433 of genotype 3a, amino acids 6-184 of genotype 4a, or amino acids 1-184 of genotype 7a, respectively (see FIG. 1). In view of the binding sites of NS5A inhibitors and the known base substitutions for resistance to the inhibitors locating in the first 100 amino acids of NS5A protein, it is inferred that the 184 amino acids at N-terminal of NS5A are sufficient to represent the characteristics of full-length NS5A of each genotype. The resultant chimera replicons could be stably transfected into Lunet cells, and all have an ability to replicate.

TABLE 1
Inhibitory activities of ASC16 in the
replicon cells of several HCV genotypes

| | Amino acid | Amino acid variation | |
|---|---|---|---|
| HCV genotype | insertion | compared with HCV 1b (%) | |
| 2a NS5A | 176 | 58 | (33) |
| 3a NS5A | 425 | 112 | (26) |
| 4a NS5A | 179 | 38 | (21) |
| 5a NS5A | 176 | 43 | (24) |
| 6a NS5A | 176 | 43 | (24) |
| 7a NS5A | 184 | 60 | (33) |

Standard 3-day assays were used to determine the inhibitory activities of ASC16 in HCV genotypes 1a, 1b, 2a NS5A, 3a NS5A, 4a NS5A, 5a NS5A, 6a NS5A, and 7a NS5A replicon cells. The inhibitor was serially diluted and used to treat the replicon cells for 3 days in the presence of 5% fetal bovine serum. The results are shown in Table 1. In these assays, the concentrations of ASC16 required to reduce the replications of HCV 1a and 1b virus by 50% (EC₅₀) and 90% (EC₉₀) were in a range of picomolar (pM); meanwhile, in these assays, the inhibition rate of ASC16 to the replication of genotype 1 virus all reached 90% at a concentration of ≤0.32 nM (0.24 ng/mL). The susceptible concentrations (EC₅₀) of ASC16 against other HCV genotypes were between 0.04 and 1.14 nM, and the EC₉₀ for all major HCV genotypes were all ≤3.35 nM (<3 ng/mL).

TABLE 2
Inhibitory effects of HCV NS5A inhibitor ASC16

| HCV | EC₅₀ (nM) | EC₅₀ | EC₉₀ (nM) | EC₉₀ |
|---|---|---|---|---|
| Genotype | Mean ± SD | (ng/mL) | Mean ± SD | (ng/mL) |
| 1a | 0.12 ± 0.01 | 0.09 | 0.32 ± 0.02 | 0.24 |
| 1b | 0.02 ± 0.002 | 0.015 | 0.03 ± 0.004 | 0.02 |
| 2a NS5A | 0.14 ± 0.03 | 0.12 | 0.45 ± 0.1 | 0.38 |
| 3a NS5A | 1.14 ± 0.3 | 0.95 | 3.35 ± 1.3 | 2.80 |
| 4a NS5A | 0.05 ± 0.01 | 0.04 | 0.13 ± 0.015 | 0.11 |
| 5a NS5A | 0.04 ± 0.01 | 0.03 | 0.12 ± 0.02 | 0.10 |
| 6a NS5A | 0.30 ± 0.1 | 0.25 | 1.1 ± 0.3 | 0.92 |
| 7a NS5A | 0.07 ± 0.02 | 0.06 | 0.2 ± 0.05 | 0.17 |

The results are the average of at least 3 independent assays.

1.2 Effect of Protein Binding

To evaluate the relative effect of serum protein binding on the efficacy of ASC16, HCV 1a and 1b replicon assays were repeated in the presence of 1% and 40% human serum proteins. The effect of human serum protein binding on the efficacy of ASC16 is represented by the fold changes of EC₅₀ and EC₉₀ assayed in the presence of human serum in a concentrate of 1%, as compared to those in the presence of human serum in a concentrate of 40%. The results for HCV 1a and 1b replicon cell lines both showed that the efficacy was reduced by an average of 9-11 folds in the presence of 40% human serum, as compared to the results obtained in the presence of 1% human serum.

1.3 Determination of Viral Specificity of ASC16

The specificity to HCV is determined by observing the potential inhibitory effects of ASC16 on one group of other viruses using a virus-cell protection assay. This group of viruses includes:

a) bovine viral diarrhea virus (BVDV), which is another member of the flaviviridae family, and originally used as an alternative model for HCV before the establishment of HCV replicon assay; both HCV and BVDV encode NS5A protein (with 15% amino acids being identical), and the protein is likely to exert a similar function during the virus replication;

b) human rhinovirus 16 (HRV-16), which is another positive-strand RNA virus and encodes a polypeptide which is subsequently cleaved into viral proteins by viral protease;

c) influenza B virus (Flu-B), which is a segmented negative-strand RNA virus.

In order to control the potential cytotoxic effects in these studies, a group of cells were co-cultured with ASC16 without viral infection, and the concentrations required to cause 50% cytotoxicity (CC₅₀) were calculated simultaneously. The results showed that ASC16 failed to show any significant inhibitory activity against any of the tested viruses (with all EC₅₀>3300 nM), and showed a significant cytotoxicity in HeLa H1A and MDCK cells at the highest test concentration (10,000 nM) (Table 3). These results confirm that ASC16 is a specific inhibitor of HCV replication.

TABLE 3
Assay of effects of ASC16 on a group
of related and unrelated viruses

| Virus | Cell line | EC₅₀ (nM) | CC₅₀ (nM) |
|---|---|---|---|
| BVDV | BT | >10,000 | >10,000 |
| HRV-16 | HeLa | >3,300 | >3,300 |
| Flu-B | MDCK | >5,000 | >5,000 |

The pharmaceutical composition of the present invention retains the advantages of ASC16 in multiple genotypes of HCV virus, with the anti-hepatitis C virus efficacy being greatly improved, and the action spectrum being wider.

### EXAMPLE 2

Assay of Combined Administration

Since HCV replicase is very error-prone, many different mutant genomic RNAs are produced every day in infected individuals. Therefore, the existing drug-resistant variants will be easily selected for clinical studies using a HCV inhibitor as monotherapy. In terms of solving the problem of drug resistance, a combined administration regimen has proven to be an effective strategy useful in the field of anti-virus. A typical example is the successful use of a three-drug combination to form a highly active antiretroviral therapy (HAART) in HIV-infected individuals. An assay of drug combination has been successfully used to evaluate the ability of combination regimen of ASC16 and ASC08, to determine whether the effect of drug combination is additive, synergistic or antagonistic.

A study on the drug combination was conducted using HCV 1b replicon cells, during which ASC16 was administrated in combination with the NS3 protease inhibitor (ASC08) in a certain concentration range (0.0625 to 16 times of EC₅₀). The calculation of combination index (CI) for drug interaction and the isobologram proposed by Chou are two basic features of the Loewe superposition model. CI is a quantitative index that reflects the degree of drug interaction in terms of synergism (CI<0.8), addition (CI=1) and antagonism (CI>1.2) for a given end point of action index. The CI calculation introduced by Chou and Talalay is based on a multi-drug action equation derived from the median-effect principle of law of mass action. Table 4 lists the CI values calculated for EC₅₀, EC₇₅ and EC₉₀ of the paired inhibitors.

TABLE 4
Summary of combination index (CI)

| Combination | Combination index (CI) ^{b} | | | |
|---|---|---|---|---|
| ratio^{a} | EC₅₀ | EC₇₅ | EC₉₀ | Note |
| ASC16 + | 1.17 ± | 1.12 ± | 1.06 ± | addition |
| ASC081: 71 | 0.08 | 0.04 | 0.04 | |

^{a}The combination ratio was calculated by dividing EC₅₀ of ASC08 by EC₅₀ of ASC16.
^{b} CI less than 0.8 indicates a synergistic activity, CI between 0.8-1.2 indicates an additive activity, and CI greater than 1.2 indicates an antagonistic activity.

The results show that when an assay is performed in HCV 1b replicon cells, a combination of ASC16 and ASC08 shows an additive effect (CI is 0.82-1.17). Although the pharmaceutical composition of the present invention (ASC16+ASC08) does not exhibit a synergistic drug efficacy, the combined use of two drugs still achieves positive effects. The studies have shown that the combined use of these two drugs is safe and feasible for the treatment of hepatitis C.

### EXAMPLE 3

Drug-Resistance Studies

Since viral RNA replicase is prone to errors during the virus replication, drug-resistant variants may have already existed and/or appear quickly in the presence of an inhibitor. The appearance of drug-resistant viruses is the main challenge in developing an effective antiviral treatment regimen for HCV infection. Therefore, understanding of drug-resistance pathway is essential to better predict the possibility of appearance of drug-resistance in patients receiving treatment and to develop treatment strategies (such as a combination therapy), which will be able to establish drug-resistance barriers. The inventors conducted a series of drug-resistance studies in order to clarify: 1) why drug-resistant variants are prone to appear in the HCV replicon cells; 2) whether amino acid substitutions occur and they can cause the reduction of susceptibility to ASC16; 3) whether the drug-resistance pathways of different virus genotypes are common or different; 4) the replication ability of emerging drug-resistant variants; 5) whether the ASC16 drug-resistant variants retain the susceptibility to other different HCV inhibitors; 6) whether the combined use of ASC16 and IFN-α or other small molecule antiviral drugs could prevent the appearance of drug-resistant variants.

Selection of Drug-Resistant Variants

Since the previous studies using both cell passaging and colony formation assay yielded the similar results (i.e. identification of major base substitutions resulting in drug resistance), a group of HCV replicon cell lines were used to screen ASC16 drug-resistant variants through a colony formation assay. Stable HCV genotype 1a and 1b replicon cell lines and stable HCV 1b chimera replicon cell lines comprising the key NS5A gene fragments of HCV genotypes 2a, 3a, 4a, 5a, and 6a were treated with ASC16 for 21 days at the concentrations equivalent to 10, 100, and 1,000 times the EC₅₀ value for their respective wild-types (WT), with the highest concentration of ASC16 being 1000 nM.

HCV 3a NS5A replicon cells and HCV 6a NS5A replicon cells were only treated with ASC16 at concentrations of 10 times and 100 times WT EC₅₀ and 1,000 nM. Due to the differences among various replicon cell lines in terms of cell growth rate, the cell seeding densities were adjusted as follows: HCV 1a, 1b, 2a NS5A and 3a NS5A replicon cell lines were 25,000 cells/dish, HCV 5a NS5A replicon cell line was 50,000 cells/dish, and HCV 4a NS5A and 6a NS5A replicon cell lines were 100,000 cells/dish. The untreated cells in a culture dish showed a confluent state on day 21 and served as a cell growth control. In order to determine whether the emerging colonies were really resistant to ASC16, the replicon RNAs were isolated from the cells in a double culture dish, amplified by RT-PCR and subjected to population sequencing.

Drug-resistant colonies appeared in most of the culture dishes treated with ASC16. As expected, there was a better correlation between the appearing frequency of colonies and the concentrations of ASC16 used (the number of colonies decreased with the increase of concentration of ASC16). The selection of 1,000 nM ASC16 failed to produce any drug-resistant colonies in HCV 1b, 3a NS5A, 5a NS5A, and 6a NS5A replicon cell lines. However, a small number of colonies did appear in HCV 1a, 2a NS5A, and 4a NS5A replicon cells that received 1,000 nM ASC16. The genotyping of replicons from the recovered colonies confirmed the presence of drug-resistant base substitutions.

There are many pathways of developing the drug resistance to ASC16, most of which involve one or more changes in the key amino acid positions 28, 30, 31 and 93 within domain 1 of NS5A protein. In the presence of lower concentrations of ASC16, other substitutions, such as P58L (4a NS5A) and T58A (6a NS5A), will also occur. However, these substitutions will not occur when the concentration of ASC16 is increased to 1,000 nM, suggesting these specific substitutions are less likely to be the major drug-resistant base substitutions. A colony formation assay using HCV 1a replicon cells found very different pathways, suggesting that a single drug-resistant pathway is not dominant, while multiple substitutions may be present in a form of combination. This is in contrast to the results obtained using HCV 2a NS5A, 3a NS5A and 6a NS5A replicon cell lines, in which the single preferred substitutions F28S (HCV2a NS5A and 6a NS5A) and Y93H (HCV3a NS5A) that can cause a high drug-resistance were found consistently. Similar to the substitutions in tandem found in HCV 1a replicon cells, the combined substitutions of L31FN+Y93H (HCV 1b), L30H+Y93H (HCV 4a NS5A) and Q30H+L31F (HCV 5a NS5A) are required to achieve a higher level of drug-resistance. Table 5 summarizes the drug-resistant base substitutions found using a set of HCV replicon cell lines representing all major HCV genotypes.

These emerging variants are similar to those previously determined with NS5A inhibitors daclatasvir and ledipasvir (GS-5885). Therefore, drug-resistant substitution is likely to be a phenomenon related to the categories rather than the compounds. Considering the nature of colony formation in these assays, the drug-resistant colonies are likely from the subpopulations of fewer cells (from 25,000 to 100,000 cells initially seeded) with the replicons that have had drug-resistant base substitutions previously.

TABLE 5
Substitutions determined by ASC16 colony formation assay

| | Selected concentrations of ASC16 | | | |
|---|---|---|---|---|
| HCV replicon | 10X WT | 100X WT | 1,000χ | |
| cells | EC₅₀ | EC₅₀ | WT EC₅₀ | 1,000 nM |
| 1a | M28T | Y93H | Q30K | M28T |
| | Q30R | — | Y93H/N | Y93N |
| | Y93C | — | — | — |
| 1b | L31V | L31V/F | L31V/F | No colony |
| | Y93H | Y93H | Y93H | — |
| 2a NS5A | F28S/V | F28S/V | F28S | F28S |
| | L31M | L31M | — | — |
| | Y93H | Y93H | — | — |
| 3a NS5A | M28T | Y93H | ND | No colony |
| | L31F | — | — | — |
| | Y93H | — | — | — |
| 4a NS5A | P58L | P58L | L30H | L30H |
| | Y93H | Y93H | P58L | Y93H |
| | — | — | Y93H | — |
| 5a NS5A | L31F | L28P | Q30H | No colony |
| | — | L31F | L31F | — |
| 6a NS5A | T58A | F28S | ND | No colony |
| | — | L31M | — | — |
| | — | P32L | — | — |

ND = not detected

Phenotypes Containing Base Substitution for Resistance to ASC16

The replicons with identified drug-resistant base substitutions were created, and the RNAs of resulting HCV replicons were then transcribed and used for a transient assay to evaluate their susceptibilities to ASC16. The susceptibility degrees of WT replicons in the transient assay were similar to the values obtained from a cell assay using the stable HCV replicons.

The drug-resistant variants appeared in the colony formation assay and their corresponding susceptibilities to ASC16 and replication ability levels are listed in Table 6. The EC₅₀ results obtained using the clonal variants have a good correlation with the screening concentrations used in the colony formation assay, because the drug-resistant variants that appeared at lower folds of EC₅₀ also had lower EC₅₀ values in the transient assay, while the variants selected at the higher concentrations also had a higher drug-resistance to ASC16.

In addition to the resistance to NS5A inhibitors almost only appearing in the regions defined by amino acids 28-31 and 93 of NS5A protein, it is also noted that there may be a diversity of amino acid variations at these specific residues, depending on the NS5A inhibitors and the HCV genotypes used during the selection. Among the 20 newly appeared variants identified and detected after the ASC16 selection, only 6 have substitutions with an EC₅₀ higher than 100 nM [Y93N (HCV 1a), L31V+Y93H (HCV 1b), F28S (HCV 2a NS5A), Y93H (HCV 3a NS5A), L30H+Y93H (HCV 4a NS5A) and F28S (HCV 6a NS5A)]. Some of substitutions [F28V and L31M (HCV 2a NS5A), L28P (HCV 5a NS5A) and L31M, P32L and T58A (HCV 6a NS5A)] only appearing at lower concentrations of ASC16 were not found at higher concentrations, and these substitutions were not tested in the transient transfection assay because they are unlikely to cause a higher degree of resistance to ASC16.

These variations did not result in a compound-specific drug resistance, because the studies on susceptibility to other NS5A inhibitors found an extensive cross resistance. Therefore, all these identified substitutions are likely to cause resistance to a certain type of drugs.

The replication ability of drug-resistant variants obtained using a larger set of HCV replicon cell lines was ranging from 1.1% to 304% of that of the parental replicon cell lines. Among 39 drug-resistant replicons produced, 29 had a replication ability which is 220% of that of WT replicons. The replication ability of HCV3a NS5A variants has been reduced to the greatest degree, and the replication ability caused by its drug-resistant substitution M28T is only 0.4% of that of WT. At the same time, L31F or Y9m substitution is not replicated at all in a transient assay (data not shown). As an unidentified compensatory substitution is likely to be required, it is impossible to evaluate HCV 3a NS5A drug-resistant variants with M28T, L31F, and Y93H substitutions. This is related to the results observed in the colony formation assay. The number of drug-resistant colonies under 10-fold EC₅₀ of WT 3a NS5A was significantly lower than that for all other genotypes tested, suggesting that the variants that appeared from 3a NS5A had a relatively poorer replication fitness. Y93H substitution was used for HCV 3a NS5A replicon cells (obtained from a cell passage assay using the NS5A inhibitor PPI-461 and confirmed that all cells contained Y93H substitution), resulting in more than 800-fold change of EC₅₀ for ASC16 (1.2 nM-1,036 nM).

There is no correlation between the degree of drug-resistance and the replication activity and/or amino acid positions. Generally speaking, the EC₅₀ concentrations caused by various substitutions in HCV 1a replicons are much higher than that caused by variation of a single amino acid in HCV 1b replicons. The substitution of residue 28 in HCV 2a NS5A and HCV 6a NS5A and the substitution of residue 93 in HCV 3a NS5A replicon cells resulted in a higher degree of resistance to ASC16, with the EC₅₀ of each genotype for ASC16 changing from a level of sub-nanomolar to micromolar. This suggests that although the substitutions that cause drug-resistance in all genotypes always appear at the key NS5A residues 28, 30, 31, and 93, the degree of drug-resistance varies, and is most likely specific to the genotypes, and depends on the existing backbone sequences.

TABLE 6
Susceptibility of drug-resistant variants of HCV selected by ASC16

| | Drug- | | | Replication |
|---|---|---|---|---|
| HCVge- | resistant | Mean | Fold | ability |
| notype | variant | EC₅₀ (nM) ±SD | change | (% of control) |
| 1a | WT | 0.03 ± 0.002 | 1 | 100 |
| | Q30R | 4.9 ± 1.3 | 168 | 58 |
| | Y93C | 5.0 ± 0.4 | 169 | 28 |
| | M28T | 5.5 ± 2.8 | 189 | 40 |
| | Y93H | 48 ± 1.0 | 1,630 | 4.8 |
| | Q30K | 87 ± 17 | 2,965 | 47 |
| | Y93N | 113 ± 7.3 | 3,876 | 22 |
| 1b | WT | 0.01 ± 0.003 | 1 | 100 |
| | L31F | 0.05 ± 0.009 | 4.0 | 162 |
| | Y93H | 0.9 ± 0.06 | 81 | 38 |
| | L31V | 0.9 ± 0.1 | 85 | 304 |
| | L31F + Y93H | 28 ± 5.4 | 2,541 | 161 |
| | L31V + Y93H | 412 ± 56 | 37,231 | 61 |
| 2a | WT | 0.12 ± 0.06 | 1 | 100 |
| | Y93H | 96 ± 23 | 816 | 31 |
| | F28S | 537 ± 19 | 4,575 | 54 |
| 3a | WT | 1.2 ± 0.3 | 1 | 100 |
| | Y93H* | 1,036 ± 85 | 863 | NA |
| 4a | WT | 0.02 ± 0.004 | 1 | 100 |
| | P58L | 3.0 ± 0.09 | 197 | 95 |
| | Y93H | 3.2 ± 0.2 | 210 | 8.0 |
| | L30H | 14 ± 0.8 | 887 | 49 |
| | L30H + Y93H | 877 ± 119 | 57,587 | 13 |
| 5a | WT | 0.08 ± 0.04 | 1 | 100 |
| | L31F | 6.1 ± 0.8 | 76 | 303 |
| 6a | WT | 0.2 ± 0.04 | 1 | 100 |
| | F28S | 422 ± 19 | 2,146 | 16 |

Each data point represents an average of ≥3 independent assays. The replication ability is related to the respective WT replicon cells(set to 100%).
* indicates the EC₅₀ produced by HCV 3a NS5A replicon cells in the cell passage assay, and the results indicate that all cells contain Y93H substitution.

Overview of Cross-Resistance

Since monotherapy with an antiviral drug will result in a higher risk of selection of drug-resistant variants, a successful long-term treatment will be likely to require a combination of inhibitors with characteristics of different and non-overlapping drug-resistance. Since ASC16 is a highly selective inhibitor of HCV (it is likely to target domain 1 region of viral NS5A protein), a series of cross-resistance assays were carried out to evaluate its ability of inhibiting the variants resistant to other different classes of HCV inhibitors. Previous studies for drug-resistance have confirmed the labeled amino acid substitutions that can cause drug-resistance and occur during cell culture and passages and/or a clinical trial for PI ASC08 (NS3 D168A). The drug-resistant HCV 1b replicons encoding these drug-resistant base substitutions were created and assayed for their susceptibilities to ASC16 and ASC08. The data in Table 7 show that ASC16 retains its ability to inhibit these drug-resistant variants, and its EC₅₀ is comparable to the result observed for WT HCV 1b replicon cells. These results confirmed the expectation of no cross resistance and the ability of ASC08 to inhibit the variants that appeared when ASC16 was used.

TABLE 7
Susceptibility of drug-resistant variants to ASC08 inhibitor

| | Mean EC₅₀ (nM) | | | | |
|---|---|---|---|---|---|
| | | | R262Q + | | |
| | | | R318W + | | |
| | | D168A | D320E | S282T | C316Y |
| | Wild-type | (NS3) | (NS5A) | (NS5B) | (NS5B) |
| ASC16 (NS5A) | 0.01 ± | 0.007 ± | 0.009 ± | 0.008 ± | 0.009 ± |
| | 0.003 | 0.002 | 0.001 | 0.0003 | 0.001 |
| ASC08 (PI) | 0.7 ± | 128 ± | 0.8 ± | 0.6 ± | 0.7 ± |
| | 0.1 | 22 | 0.08 | 0.08 | 0.08 |

The mean value represents the average of 3 independent assays.

In order to evaluate whether the ASC16-resistant variants retain their susceptibility to ASC08, the HCV 1a resistant variant containing L31V+Y93H substitutions was transiently transfected into Huh7-Lunet cells and the EC₅₀ of ASC08 was calculated. As expected, no cross-resistance was observed. At the same time, a reduction of the susceptibility to ASC16 was only observed for HCV 1a L31V+Y93H replicon (see Table 8).

TABLE 8
Susceptibility of ASC16-resistant variants to ASC08

| HCV 1a | Mean EC₅₀ (nM) ± SD | | | | |
|---|---|---|---|---|---|
| variant | ASC16 | ASC08 | HCV-796 | PSI-6130 | Cyclosporine A |
| Wild-type | 0.03 ± | 5.4 ± | 11.8 ± | 770 ± | 359 ± 14 |
| | 0.002 | 0.9 | 0.9 | 139 | |
| L31V + | 1,160 ± | 4.1 ± | 11.4 ± | 548 ± | 341 ± 16 |
| Y93H | 16 | 0.1 | 3.3 | 64 | |

The mean value represents the average of >3 independent assays.

These results suggest that the ASC16-resistant variants retain their susceptibility to ASC08. Therefore, ASC16 can be combined with ASC08 inhibitor to prevent the appearance of drug-resistance virus strains.

What is claimed is:

1. A pharmaceutical composition, characterized in that said pharmaceutical composition comprises ravidasvir or a pharmaceutically acceptable salt thereof and danoprevir or a pharmaceutically acceptable salt thereof; preferably, said pharmaceutically acceptable salt of ravidasvir is ravidasvir hydrochloride, and said pharmaceutically acceptable salt of danoprevir is danoprevir sodium.

2. The pharmaceutical composition according to claim 1, characterized in that the mass ratio of ravidasvir or a pharmaceutically acceptable salt thereof to danoprevir or a pharmaceutically acceptable salt thereof is 1:1.

3. The pharmaceutical composition according to claim 1, characterized in that said pharmaceutical composition further comprises ritonavir or a pharmaceutically acceptable salt thereof; preferably, the mass ratio among ravidasvir or a pharmaceutically acceptable salt thereof, danoprevir or a pharmaceutically acceptable salt thereof, and ritonavir or a pharmaceutically acceptable salt thereof is 1:1:1.

4. The pharmaceutical composition according to claim 1 or 2, characterized in that ravidasvir or a pharmaceutically acceptable salt thereof and danoprevir or a pharmaceutically acceptable salt thereof are placed separately; preferably, the administration dosages of danoprevir or a pharmaceutically acceptable salt thereof and ravidasvir or a pharmaceutically acceptable salt thereof are: ravidasvir or a pharmaceutically acceptable salt thereof, once a day, 200 mg; danoprevir or a pharmaceutically acceptable salt thereof, twice a day, 100 mg each time, respectively.

5. The pharmaceutical composition according to claim 3, characterized in that ravidasvir or a pharmaceutically acceptable salt thereof, danoprevir or a pharmaceutically acceptable salt thereof, and ritonavir or a pharmaceutically acceptable salt thereof are placed separately; preferably, the administration dosages of danoprevir or a pharmaceutically acceptable salt thereof, ravidasvir or a pharmaceutically acceptable salt thereof, and ritonavir or a pharmaceutically acceptable salt thereof are: ravidasvir or a pharmaceutically acceptable salt thereof, once a day, 200 mg; danoprevir or a pharmaceutically acceptable salt thereof, twice a day, 100 mg each time; ritonavir or a pharmaceutically acceptable salt thereof, twice a day, 100 mg each time, respectively.

6. The pharmaceutical composition according to any one of claims 1 to 5, characterized in that said pharmaceutical composition further comprises a pharmaceutically acceptable excipient;
preferably, said excipient is selected from one or more of poloxamer, mannitol, microcrystalline cellulose, isomalt, Span 20, or polyvinylpyrrolidone.

7. The pharmaceutical composition according to any one of claims 1 to 6, characterized in that the dosage form of said pharmaceutical composition is a film-coated tablet.

8. A kit for treating viral hepatitis C, characterized in that said kit contains one unit dose of ravidasvir or a pharmaceutically acceptable salt thereof, one unit dose of danoprevir or a pharmaceutically acceptable salt thereof, and another unit dose of danoprevir or a pharmaceutically acceptable salt thereof, which are placed separately; preferably, said one unit dose of ravidasvir or a pharmaceutically acceptable salt thereof is 200 mg of ravidasvir or a pharmaceutically acceptable salt thereof, and said one unit dose of danoprevir or a pharmaceutically acceptable salt thereof and another unit dose of danoprevir or a pharmaceutically acceptable salt thereof are 100 mg of danoprevir or a pharmaceutically acceptable salt thereof, respectively.

9. The kit according to claim 8, characterized in that said kit further comprises one unit dose of ritonavir or a pharmaceutically acceptable salt thereof and another unit dose of ritonavir or a pharmaceutically acceptable salt thereof, which are placed separately; further preferably, said one unit dose of ritonavir or a pharmaceutically acceptable salt thereof and another unit dose of ritonavir or a pharmaceutically acceptable salt thereof are 100 mg of ritonavir or a pharmaceutically acceptable salt thereof, respectively.

10. Use of the pharmaceutical composition according to any one of claims 1 to 7 in the preparation of a therapeutic drug for hepatitis C.

11. A method for treating hepatitis C, characterized in that said method comprises administering a therapeutically effective amount of the pharmaceutical composition according to any one of claims 1 to 7 to a subject in need thereof, or applying the kit according to claim 8 or 9 to a subject in need thereof.
